(19) United States
(12) Patent Application Publication
(10) Pub. No.: US 2012/0021476 A1
(43) Pub. Date: Jan. 26, 2012

(54) Trace Element Solution For Biogas Methods

(75) Inventors: Hans Friedmann, Scheyern-Fernhag (DE); Jürgen Kube, Pfaffenhofen (DE)

(73) Assignee: AGRAFERM TECHNOLOGIES AG, Pfaffenhofen/Ilm (DE)

(21) Appl. No.: 12/808,438

(22) PCT Filed: Dec. 19, 2008
(86) PCT No.: PCT/EP2008/068115

§ 371 Date: Jan. 24, 2011

(30) Foreign Application Priority Data
Dec. 19, 2007 (DE) 10 2007 061 138.4

(51) Int. Cl. C12P 5/02

(52) U.S. Cl. 435/167

(57) ABSTRACT

The invention relates to a trace element solution for the supplementing of nutrients for an anaerobic fermentation, in particular a biogas process, comprising at least one trace element and at least two complexing agents. Complexing agents are used which (1) are able to transport the trace elements in complexed form across the cell membrane and which (2) release the trace elements in the cell. Where applicable, the complexing agents are biologically decomposable.

### TECHNICAL FIELD

The invention relates to additives for anaerobic fermentation, in particular processes for the production of biogas, which improve the availability of trace elements for the microorganisms.

### PRIOR ART

Biogas is a mixture of the main components methane and CO₂. In addition it contains small amounts of water vapour, H₂S, NH₃, H₂, N₂ and traces of low fatty acids and alcohols.

In a biogas plant, substrates are fermented to biogas (CO₂ and CH₄) under oxygen exclusion. This fermentation is divided into four stages: the fermentative phase, in which the large biopolymers are dissolved, the acidogenic phase, in which the dissolved monomers and oligomers are converted into organic acids, alcohols, CO₂ and hydrogen, the acetogenic phase, in which the organic acids and alcohols are converted into acetic acid, hydrogen and CO₂ and finally the methanogenic phase, in which methane is formed from acetic acid or CO₂ and hydrogen. In addition, the reduced, partly water-soluble end products NH₃ and H₂S are also produced in the biogas process.

The microorganisms required for this purpose catalyse the necessary conversion reactions through enzymes. Many enzymes, in particular the enzymes responsible for regulation of the reduction-equivalent household, require metal ions as co-enzymes.

The hydrogenases (EC 1.12.x.x) may be cited as an example. Hydrogenases catalyse the reaction:

2H⁺+electron donor [symbol] H₂+electron acceptor

They are thus involved in hydrogen production, a very important step in the biogas process. Besides co-substrates such as FAD(H), NAD(P)(H) or ferredoxin, which may also contain trace elements (e.g. Fe), these enzymes require the co-factors Ni (e.g. EC1.12.1.2), Fe—S compounds (e.g. EC1.12.5.1) or Se (e.g. EC1.12.2.1).

Another important enzyme in the methane synthesis, which requires trace elements (in particular Co), is the acetyl-CoA:corrinoid protein O-acetyltransferase (EC2.3.1.169), which allows acetyl-CoA to react to a methyl group, carbon monoxide and CoA, e.g. in methaneosarcina barkeri.

The provision of the microorganisms in the biogas process with the essential trace elements (micro-nutrients) is inhibited by the presence of H₂S, which dissociates to 2H++S²⁻. Many of the important trace elements form sulphides which are not easily dissolved, as soon as even only small amounts of H₂S are in solution. For example given the following assumptions: pH7, 37° C., 500 ppm H₂S in the biogas, m(S)>>m(Ni), ideal mixing, equilibrium between gas and liquid phases, c(Ni)=5 μmol/L only 3×10⁻¹⁷ mol/L, i.e. 0.000,000,001% of the nickel is in aqueous solution and therefore free and bio-available. In the case of copper, the sulphide precipitation is even so strong that, under the assumptions as given above, a 1000 m³ reactor contains in terms of figures only 10⁻⁵ Cu²⁺ ions; the copper is therefore not bio-available.

The anion of the carbon dioxide (CO₃²⁻) forms compounds which are hard to dissolve especially with representatives of the rare earths. Since the gas phase of an anaerobic reactor may contain up to 50% CO₂ and in addition there is also often a mass transfer limitation of the CO₂ from the liquid phase into the gas phase and an increased hydrostatic pressure at the bottom of tall reactors, the precipitation reactions of the carbonate play an important role in the bio-availability of the Ca²⁺ and Mg²⁺.

In published patent specification DE10300082A1 the addition of a trace element solution to an anaerobic reactor is disclosed. The trace elements are fed to the reactor as sulphate, chloride, selenate or molybdenum salts in aqueous solution, without regard to the bio-availability of the trace elements. In the presence of H₂S the majority (>99.9%, see above) of the ions able to precipitate do so as sulphides. The nature of the anions of the trace element salt is not important for the bio-availability of the trace element concerned.

Commercially available trace element compositions, which are used as supplements for substrates, in particular of vegetable agricultural raw materials or industrial effluents, are used in quantities of approx. 1-2 kg/tonne of dry substance of the substrates. Because of the heavy sulphide precipitation of the metals during fermentation, the fermentation residue may not be used as fertiliser, since the permitted metal concentration for fertiliser is far exceeded.

In order to increase the solubility of the trace elements, they may be added in an acid solution. Due to the lower pH value, the dissociation equilibrium of H₂S and S²⁻ is shifted to H₂S, thus preventing precipitation. The precipitation of not-easily-dissolved hydroxide salts is also prevented in this way. After introduction into the biogas reactor, however, the trace elements thus dissolved once again precipitate as sulphides, since the pH value in a biogas reactor is for example 6-8.

A further possible means of making trace elements bio-available is to immobilise them on organic carrier materials (DE10139829A1), cereal extrudates (DE10226795A1) shaped mineral bodies (EP0328560B1) or zeolites (AT413209B). This form of presentation has the advantage that the microorganisms settle on the carriers and the required trace elements are able to diffuse out of the carriers into the microorganisms without being precipitated. A disadvantage of this method is that it is possible only with solid suspensions of low concentration and at low levels of viscosity. In a bioreactor with high solid concentrations, in which mass transfer phenomenon play an important role, the microorganisms cannot be supplied in this way. Moreover anaerobic cultures tend to form very stable bio-films which, over the course of time, would represent a transfer resistance factor. It should also be mentioned that some anaerobic bacteria (e.g. cellulose-decomposing clostridia) must settle directly on the substrate on the carrier materials in order to digest it. An additional supply to these cells of trace elements on fixed carriers is therefore hardly possible.

The levels of efficiency of these dosage methods are however low, i.e. only a fraction of the dosed trace elements are actually made use of in the biogas production. The overwhelming majority of trace elements precipitates as sulphide in the sludge or remains in heavily complexed form in the liquid fermentation residue. The solid and liquid fermentation residues are intended for application to the fields as fertiliser, on which the future substrates for biogas plant grow. A steady supplementing of the biogas reactor with large amounts of trace elements would lead to an accumulation of the trace elements which are toxic in high concentrations. An improvement in the form of presentation of the trace elements would reduce the quantity of trace elements required and thus also the heavy metal load in the fermentation residues.

From U.S. Pat. No. 5,342,524 it is known that, with the addition of certain complexing agents to a substrate for anaerobic biogas fermentation, the solubility of the trace elements in the fermentation broth is increased, and that by this means the methane yield may be significantly improved.

### DESCRIPTION OF THE INVENTION

The problem of the invention is to provide trace elements for anaerobic fermentation, in particular for a biogas process, in an improved formulation, which is stable relative to interfering substances such as Fe(III), and, where applicable impact loads, and which enhances the bio-availability of the trace elements and therefore their conversion by the microorganisms present in the bioreactor; while in the fermentation residue the permitted limits for heavy metal concentrations in the fertiliser should not be exceeded. The problem is solved by the subjects defined in the patent claims.

“Bio-availability” is to be understood as meaning the amount and/or the form of presentation of a trace element which can be resorbed by the microorganisms in the bioreactor. Preferably this involves a form or compound of the trace element which is soluble under the conditions of fermentation, i.e. it is not precipitated

The invention relates to a trace element solution for the supplementing of trace elements in anaerobic fermentation, in particular for methods of producing biogas which are carried out under neutral or weak acid conditions in which trace elements may precipitate, for example as sulphide salts.

Besides at least one, preferably several, trace elements the solution includes complexing agents. Complexing agents are compounds suitable for the complexing and masking of metals. Some are also known by the name of “chelating agent”. The complexing occurs through a coordinative bond between the metal atom and one or several molecules, i.e. ligands, of the complexing agent, which surround the metal atom. The complexing constants of the complexing agent according to the invention must be high enough to maintain the solubility of the respective trace elements of the solution according to the invention in the presence of the sulphide ions in the fermenter, taking into account the pH value and the dissociation constants of the complexing agent and of the H₂S.

A trace element will not precipitate with an appropriate, present anion (e.g. S²⁻, CO₃²⁻ or OH⁻), if the following condition is satisfied:

$${K}_{L} ( \frac{{H}^{+} + {K}_{a}}{{K}_{a}} - 1 ) \geq \frac{{K}_{SP}}{A}$$

K_{L} Stability constant of the complex
H⁺ concentration
Kₐ Dissociation constant of the complexing agent
K_{SP} Solubility product
A Anion concentration (S²⁻, CO₃²⁻, OH⁻)=f(pH), increases as pH rises

Preferably the solution includes complexing agents and trace elements in at least equimolar amounts, so that the majority of added trace elements in the fermenter are largely present as complexes. If necessary, the complexing agents according to the invention may be present in excess in the trace element solution. The excess of complexing agents according to the invention may be a multiple of the trace element solution, so that metal ions escaping from the substrate (e.g. Mg²⁺, Ca²⁺) or fed into the bioreactor (e.g. Fe³⁺) may also be complexed.

The inventors have found that, in an exemplary reference system of water-EDTA-Fe²⁺—Ni²⁺—Co²⁺—H₂S, a strong complexing agent such as EDTA (ethylenediaminetetraacetic acid) complexes trace elements in equimolar amounts, even though at neutral pH values only a small portion of the EDTA is present as active EDTA⁴⁻-anions in a water-EDTA-mixture. Even the presence of H₂S causes no precipitation in the presence of EDTA. If instead of EDTA, complexing agents are used which form complexes of two or several ligands of the complexing agent per metal atom, then a correspondingly multiple (double, multiple) molar amount of the relevant ligands must be used in order to complex the trace elements in the solution.

If now the amount of the complexing agent (e.g. EDTA) is reduced in stages, then in the sequence of the complexing constants (pK) first Fe²⁺ (pK=14.3), then Co²⁺ (pK=16.3), and finally Ni²⁺-ions (pK=18.6) are precipitated. The same applies when an interfering substance is added (such as e.g. Fe³⁺, pK=25.1; Mn³⁺; Hg²⁺), which enters into stronger bonds with the complexing agent and displaces metals with lower complexing constants. An objective of the invention is to formulate the trace element solution according to the invention in such a way that even with such interference by various metal ion species (e.g. Fe³⁺, Mg²⁺), an adequate amount of ions of the other metal ions of the trace element solution remains complexed in solution to exclude any limitation of these ions, in particular Co²⁺, Ni²⁺ or Mn²⁺, during fermentation.

By partial replacement of the strong complexing agent such as e.g. EDTA by a mixture of two different complexing agents with different affinities, i.e. complexing constants, to the metals, the majority of trace elements may be complexed completely under the conditions of a biogas fermentation and moreover partly complexed trace elements will still be available to the system even in the event of interference by a metal species such as Fe³⁺, Mg²⁺ or Ca²⁺. Only a portion of the metal species concerned is then precipitated by the sulphide into the fermentation broth. Consequently one embodiment of the invention is a solution with trace elements, which includes at least two different complexing agents, wherein the complexing agents differ in the complexing constants or affinities to metal ions. I.e. preferably different complexing agents are selected, which complex the metal ions to different degrees, while they should be strong enough to prevent precipitation under the conditions of biogas fermentation. If necessary, the solution according to the invention may also contain three, four, five or more complexing agents. Through the use of two or more different complexing agents, the effectiveness of the trace element presentation is increased and a form of presentation for the trace elements is obtained which remains stable even under fluctuating reaction conditions. For, if a metal species is displaced from a complex by another metal species, which has a greater affinity (pK) to this complexing agent, the displaced metal species will then form a new complex with a second complexing agent. The use of at least two different complexing agents also makes possible the increased availability of difficult-to-dissolve micronutrients such as cobalt, nickel or manganese in a biogas fermentation despite the high load of sulphide and carbonate ions.

Moreover, through the use of least two different complexing agents according to the invention, the bio-availability in particular of cobalt, nickel, zinc and manganese is significantly increased and the yield of the biogas process is greatly improved, since in particular cobalt and nickel are essential for the methaneogenesis. An especially advantageous feature is that the bio-availability of cobalt is increased many times over with the trace element solution according to the invention. At the same time, due to the increase in bio-availability and with it of solubility, the necessary amount of trace elements for a corresponding rise in the efficiency of the process is very much reduced. So, just the addition of trace element solution according to the invention of, for example, 30 mL/tonne of dry substance of the fermentation substrates may be enough for the supplementation, in particular of a mono-substrate.

Table 1 shows how, with the same dosage of trace elements, their concentration and/or bio-availability is improved by the solution according to the invention: if the trace elements are complexed by the chelate complexing agent NTA, with NTA being added in a stoichiometric amount of 50% of the total amount of trace elements in a trace element solution (as in the description of the known trace element solution for medium 141 of the DSMZ (German Collection of Microorganisms and Cell Cultures); see Table 5), the cobalt, nickel and zinc are hardly bio-available at all. Only 2 ppm of the added volume of nickel is available. Even with the sole use of an excess stoichiometric amount of 500% citrate (5 times the stoichiometric amount as compared with the trace element solution) the bio-availability of essential trace elements such as cobalt or nickel is not improved. On the other hand, if a trace element solution according to the invention with two complexing agents is used, namely EDTA and a mixture of phosphoric acids, then cobalt, nickel and manganese are 100% bio-available. In Table 1 the trace element solution according to the invention contains the complexing agent in a stoichiometric amount of 60% EDTA and 60% of a phosphoric acid mixture relative to the overall amount of trace elements, which is composed of identical molar amounts of pyrophosphoric acid (H₄P₂O₇), polyphosphoric acid (H₆P₄O₁₃), metaphosphoric acid (H₄P₄O₁₂), hypophosphoric Acid (H₃PO₂) and phosphorus acid (phosphonic acid) (H₃PO₃).

Since, through the use of two or more different complexing agents, the bio-availability of cobalt, nickel and zinc may be so increased, the dosage of these metals in the trace element solution may be distinctly reduced.

TABLE 1
Concentrations of the trace elements in mol/L

| | | | | | | Invention | |
|---|---|---|---|---|---|---|---|
| | | | | | | Fermentation | |
| | | Fermentation | Fermentation | Fermentation | | residue | |
| | Solution, | residue | residue | residue | | 60% EDTA | |
| | without | without | with | citrate | Bio- | 60% phosphoric | Bio- |
| | complex | complex | 50% NTA¹⁾ | (500%)²⁾ | availability | acids³⁾ | availability |
| Fe | 2.0E−06 | 4.3E−10 | 2.0E−06 | 2.0E−06 | 100% | 2.0E−06 | 100% |
| Mg | 1.4E−04 | 1.4E−04 | 1.4E−04 | 1.4E−04 | 100% | 1.4E−04 | 100% |
| Ca | 5.0E−06 | 5.0E−06 | 5.0E−06 | 5.0E−06 | 100% | 5.0E−06 | 100% |
| Cu | 2.2E−07 | 6.9E−37 | 8.2E−30 | 5.1E−22 | 0% | 2.5E−22 | 0% |
| Co | 3.4E−06 | 6.9E−15 | 8.2E−11 | 5.1E−08 | 0% | 3.4E−06 | 100% |
| Ni | 4.7E−07 | 8.7E−19 | 1.0E−12 | 6.4E−12 | 0% | 4.7E−07 | 100% |
| Zn | 3.1E−06 | 9.5E−17 | 1.1E−11 | 7.0E−10 | 0% | 3.1E−06 | 100% |
| Mn | 2.3E−05 | 6.1E−08 | 7.2E−06 | 4.5E−06 | 31% | 2.3E−05 | 100% |
| Al | 3.2E−07 | 3.2E−07 | 3.2E−07 | 3.2E−07 | 100% | 3.2E−07 | 100% |

¹⁾0.5 times stoichiometric amount NTA
²⁾5 times stoichiometric amount citrate
³⁾0.6 times stoichiometric amount EDTA and 0.6 times stoichiometric amount of the phosphoric acid mixture

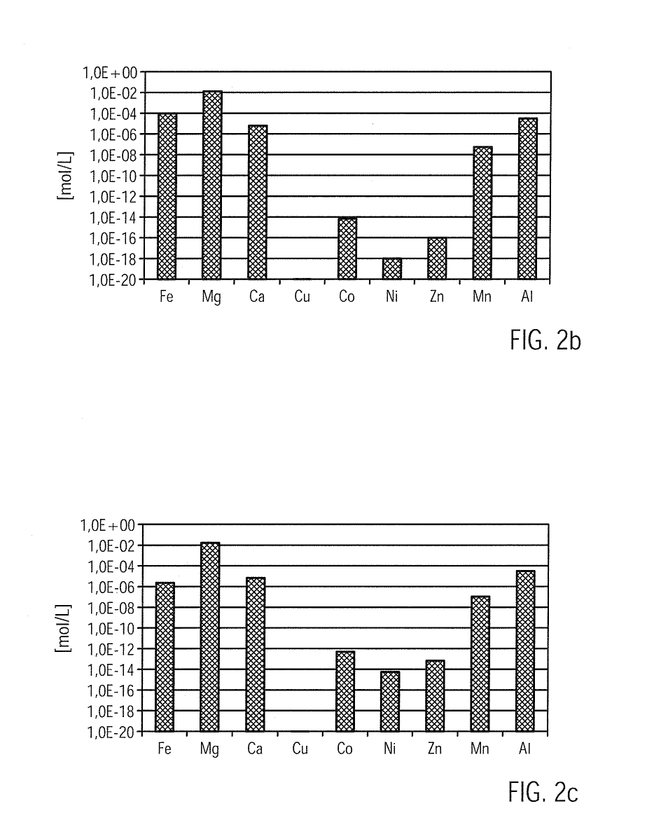

Table 2, left-hand column, shows that bio-availability of the trace elements cannot be improved, if their concentration in the trace element solution by a complexing agent (NTA) is increased 100 times. On the contrary, the bio-available content of cobalt, nickel and manganese declines. Cobalt, nickel and manganese are namely displaced from the complexes by iron, the concentration of which similarly increases. (Cobalt, nickel and manganese have a lower complexing constant than iron). There is then no longer any complexing agent remaining which is able to complex nickel, cobalt and manganese. This is shown in FIGS. 2a and 2b.

Even if, in a trace element solution with a complexing agent (NTA), the iron content is held constant and only the concentration of the other trace elements is increased 100 times, the bio-available content of cobalt, nickel and manganese falls, even though these trace elements have a higher complexing constant than calcium or magnesium. This is shown in Table 2, right-hand column and FIG. 2c. Cobalt, nickel and manganese are in fact less soluble than calcium or magnesium. Since the concentration of the free Mg²⁺ is roughly 17 orders of magnitude greater than the concentration of free Ni²⁺, the complex is formed between NTA and magnesium, even though the complexing constant Ni at pK=12 is much higher than for magnesium at pK=6. This phenomenon occurs especially with anaerobic biogas fermentation, since here large concentrations of CO₃²⁻ and S²⁻ develop. With a trace element solution according to the invention, on the other hand, the bio-availability of cobalt, nickel and manganese are significantly improved and these metals are also held adequately in solution even under heavy exposure to CO₃²⁻ and S²⁻.

TABLE 2
Concentrations of the trace elements in mol/L

| | | | | Fermentation |
|---|---|---|---|---|
| | | | | residue with |
| | Fermentation residue with 50% NTA¹⁾ | | | 50% NTA¹⁾ |
| | | 100 × times | | 100 × times |
| | Simple²⁾ | metal | Difference | metal⁴⁾ 1 × Fe |
| Fe | 2.00E−06 | 8.72E−05 | 4260.1% | 2.00E−06 |
| Mg | 1.40E−04 | 1.37E−02 | 9698.1% | 1.37E−02 |
| Ca | 5.00E−06 | 6.91E−06 | 38.2% | 6.95E−06 |
| Cu | 8.18E−30 | 6.94E−37 | −100.0% | 4.28E−32 |
| Co | 8.18E−11 | 6.94E−15 | −100.0% | 4.35E−13 |
| Ni | 1.02E−12 | 8.68E−19 | −100.0% | 5.36E−15 |
| Zn | 1.12E−11 | 9.55E−17 | −100.0% | 5.90E−14 |
| Mn | 7.22E−06 | 6.07E−08 | −99.2% | 9.82E−08 |
| Al | 3.20E−07 | 3.21E−05 | 9931.3% | 3.21E−05 |

¹⁾Trace elements with 0.5 times stoichiometric amount NTA complexed as in Table 1
²⁾Trace element composition as in Table 5
³⁾100 times the trace element amount of Table 5
⁴⁾100 times the amount of Mg, Ca, Cu, Co, Ni, Zn, Mn, Al and 1 times the amount of Fe compared with the amount in Table 5

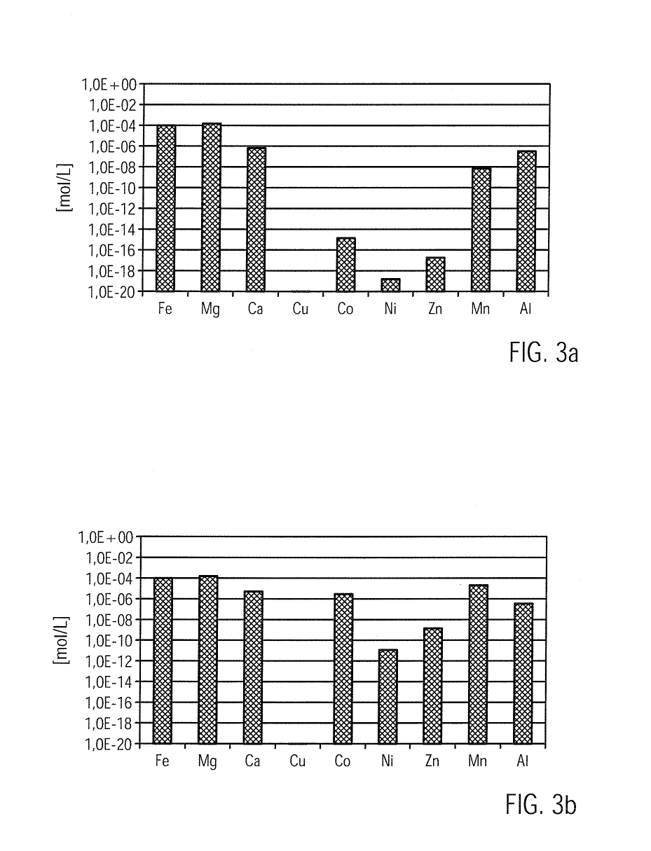

Table 3 and FIG. 3 show how interfering agents (Fe(III)) affect the concentration and bio-availability of the trace elements in a solution with only one complexing agent. As soon as larger amounts of Fe(III) from the fermentation substrate reach the fermentation broth then, with a trace element solution which comprises only one complexing agent, precipitation of the other trace elements occurs, since Fe(III) has a greater affinity to the complexing agent and the other trace elements are displaced from the complexes, whereupon the trace element precipitates. If on the other hand a trace element solution according to the invention with at least two different complexing agents is used, then the bio-availability of the trace elements for biogas fermentation is maintained. Table 3 and FIG. 3b show the extent to which, in this example, the bio-availability of the trace elements according to the invention is improved, even with the addition of an interfering agent (Fe(III)) as compared with solutions containing only one complexing agent (NTA).

TABLE 3
Concentrations of trace elements in
the fermentation residue in mol/L

| | 50% | 50% | Invention | |
|---|---|---|---|---|
| | NTA¹⁾ | NTA¹⁾ | 60% EDTA + | 100% EDTA + |
| | no | 500% | 60% P-Mix³⁾; | 1000% P-Mix⁴⁾, |
| | Fe(III) | Fe(III)²⁾ | 500% Fe(III) | 500% Fe(III) |
| Fe | 2.0E−06 | 8.9E−05 | 1.0E−04 | 1.0E−04 |
| Mg | 1.4E−04 | 1.4E−04 | 1.4E−04 | 1.4E−04 |
| Ca | 5.0E−06 | 6.4E−07 | 5.0E−06 | 5.0E−06 |
| Cu | 8.2E−30 | 1.2E−37 | 3.1E−36 | 1.1E−21 |
| Co | 8.2E−11 | 1.2E−15 | 3.4E−06 | 3.4E−06 |
| Ni | 1.0E−12 | 1.5E−19 | 1.1E−18 | 1.4E−11 |
| Zn | 1.1E−11 | 1.7E−17 | 9.6E−17 | 1.5E−09 |
| Mn | 7.2E−06 | 7.3E−09 | 2.5E−07 | 2.3E−05 |
| Al | 3.2E−07 | 3.2E−07 | 3.2E−07 | 3.2E−07 |

¹⁾0.5 times the stoichiometric amount of NTA (50% of the molar total amount of trace elements)
²⁾0.5 times the stoichiometric amount of NTA(50%), addition of 5 times the stoichiometric amount (500%) of Fe (III) (500% of the molar total amount of trace elements)
³⁾0.6 times the stoichiometric amount of EDTA (60%) and 0.6 times the stoichiometric amount of the mixture of phosphoric acids (=60% P-Mix) of the phosphoric acid mixture of Table 1
⁴⁾simple stoichiometric amount of EDTA (100%) and ten times stoichiometric amount of the mixture of phosphoric acids (=1000% P-Mix) of the phosphoric acid mixture of Table 1 relative to the total amount of trace elements

Strong complexing agents such as for example EDTA or NTA are able to complex completely all trace elements of a trace element solution with the exception of copper. If however only one complexing agent is used in the trace element solution, then the addition of Fe(III) leads to recomplexing; e.g. FeCl₃ to the desulphurisation of the bioreactor or Fe(III) bound in vegetable substrates. In the course of this, the Fe(III) dissolves the EDTA from the trace element and is then present as complexed Fe-EDTA. The trace element precipitates. A similar phenomenon occurs with calcium and magnesium due to the low solubility of the essential trace elements cobalt, nickel and manganese, in which case the macro-elements calcium and magnesium drive the micro-elements cobalt, nickel and manganese out of the complexes.

Thus, one embodiment of the invention is a trace element solution with at least two complexing agents, which differ in the complexing constants (pK) for Fe³⁺. Fe³⁺ is then complexed with the complexing agent to which it has a higher affinity (pK). The one or more other complexing agents is or are then available for complexing the other trace elements. The complexing agents are therefore chosen so that at least one first complexing agent Fe³⁺ is able to complex in a stable manner, and at least one second complexing agent can complex the other trace elements under conditions (pH-value, [S²⁻], [CO³⁻]) of a biogas fermentation; even in the presence of fermentation substrates which are rich in Ca²⁺ and/or Mg²⁺. The trace element solution according to the invention also improves the bio-availability of the trace elements in other types of anaerobic and aerobic fermentation, in particular in processes with conditions under which trace elements may precipitate.

Preferably therefore the trace element solution according to the invention comprises a first complexing agent with a greater complexing constant (pK) for Fe³⁺, than for other trace elements, in particular Co²⁺ or Ni²⁺, and a second complexing agent with affinities or complexing constants for trace elements which are satisfactory for complexing the trace elements under the condition of biogas fermentation sufficiently that they are adequately bio-available and, preferably their precipitation is largely avoided. For this purpose the complexing constant of the second complexing agent for the trace element concerned should be advantageously at least pK=2, preferably pK=5-10 and especially preferably pK≧10. Naturally as second complexing agent a mixture of two, three or several complexing agents may also be used, with each complexing agent having at least to one of the trace elements in the trace element solution a complexing constant of pK=2, preferably pK=5-10 and especially preferably pK≧10. For example as second complexing agent a complexing agent is chosen which has a complexing constant (pK) for Co²⁺ and/or Ni²⁺of pK=2, preferably pK=5-10 and especially preferably pK≧10.

Preferably the complexing constant (pK) for Fe³⁺ of the second complexing agent is smaller (weaker) than the complexing constant (pK) for Fe³⁺ of the first complexing agent. Especially preferred is for the first complexing agent to be a strong complexing agent with a complexing constant (pK) for Fe³⁺ of pK=10,preferably pK≧20, especially preferably pK≧20. Where applicable, the complexing constants (pK) for Fe³⁺ of the first and second complexing agents may differ from one another by at least 2, 3, 4 or 5 times.

The complexing agents may be present in different amounts in the trace element solution. Preferably there is at least an equimolar amount of complexing agent relative to the trace elements. Also advantageous is the addition of the complexing agent in excess of the trace elements, for example 10, 30, 50, 100 or more than 1000 times, depending on the fermentation substrate used and on the conditions of fermentation (e.g. addition of FeCl₃ for desulphurisation). The proportions of the different complexing agents relative to one another may also vary over a very wide range. For example it may be advantageous to use a weaker complexing agent or complexing agent mixture (e.g. phosphoric acid mixture) in a multiple, e.g. 5-5000 times, preferably 50-2000 times, especially preferably, 100-1500 times the molar amount of a stronger complexing agent (e.g. tertiary amine), in order to optimise the bio-availability of the trace elements and/or stability of the complexing.

Within the scope of the application, the term complexing constant is used to mean the same as complex stability constant or complex association constant and results from the product of the individual equilibrium constants of the reactions during complexing. K=[MLₙ]/[M][L]ⁿ, wherein [MLₙ] is the molar equilibrium concentration of the metal complex, [M] the molar equilibrium concentration of the free metal ions, [L] the molar equilibrium concentration of the ligand and n the number of ligands bound in the complex. In this application, the pK value is given as the value of the stability constant.

Preferably the complexing agents used have a complexing constant (pK) of at least 5, preferably at least 10, especially preferably at least 20 for at least one, preferably all, metal ion(s) of the trace element solution and, if necessary are anaerobically decomposable.

The complexing properties of exemplary complexing agents with selected bivalent and trivalent metal ions are listed in Table 4, wherein “+++” stands for excellent (pK>20), “++” for very good (pK=10-20), “+” for good (pK=5-10), “0” for moderate (pK=2-5), “−” for poor (pK=0-2) complexing and “f” for precipitation.

TABLE 4

| Complexing agents | Mg++ | Ca++ | Fe++ | Mn++ | Co++ | Ni++ | Cu++ | Al+++ | Fe+++ | Zn++ |
|---|---|---|---|---|---|---|---|---|---|---|
| H4Fe(CN)6 | 0 | 0 | | | | | | | | f |
| Fe(CN)6— | 0 | 0 | | | | | | | 0 | |
| HCN | | | +++ | | | +++ | | | +++ | f |
| HNCS | | | − | − | − | − | f | | + | |
| NH3 | f | f | | − | + | + | ++ | | | + |
| H3PO2 | | | | | | | | | + | − |
| H3PO3 | | | | | | 0 | f | | + | |
| H3PO4 | f | f | f | | 0 | 0 | 0 | | f | f |
| H4P2O7 | +++ | f | | +++ | + | + | ++ | | | + |
| H5P3O10 | + | + | + | + | + | + | + | | | + |
| H6P4O13 | + | + | | | | | ++ | | | |
| H4P4O12 | + | + | | + | 0 | 0 | + | | | + |
| Cl— | − | − | − | − | − | − | f | | − | f |
| CH2OOH | − | − | | | − | − | 0 | | ++ | − |
| C2H4OOH | − | − | − | − | − | − | 0 | − | + | − |
| C2H5OOH | − | − | − | − | − | − | 0 | − | 0 | − |
| C3H7OOH | − | − | − | − | − | − | 0 | − | 0 | − |
| (CH3)2CHCOOH | | | | | | | 0 | | 0 | |
| C4H9OOH | | | | | | | 0 | | | |
| (CH3)2C2H3OOH | | | | | | | 0 | | | |
| H2PO3(CH2)2COOH | 0 | 0 | | 0 | 0 | 0 | | | | 0 |
| C3H7O7P | 0 | 0 | | 0 | 0 | 0 | | | | 0 |
| CH2(OH)COOH | − | − | − | − | 0 | 0 | 0 | | 0 | 0 |
| CH3CH(OH)COOH | − | − | | − | 0 | 0 | 0 | | | 0 |
| CH3CH2CH(OH)COOH | | | | | − | 0 | 0 | | | 0 |
| C6H10O7 | | | ++ | | | | | | | |
| C2H2O3 | | | | | | − | | | | − |
| CH3(C═O)COOH | | − | | − | | − | 0 | | | |
| CH2(SH)COOH | | | ++ | + | ++ | +++ | | | | +++ |
| CH3CH(SH)COOH | | | | | | +++ | | | | +++ |
| C5H4O4N2 | | | | | + | + | + | | | |
| (COOH)2 | 0 | f | + | 0 | + | + | + | | ++ | + |
| HOOCCH2COOH | 0 | 0 | | | + | 0 | + | ++ | + | + |
| HOOC(CH2)2COOH | 0 | − | − | 0 | 0 | 0 | 0 | | + | 0 |
| HOOCCH(OH)COOH | 0 | 0 | | | 0 | 0 | + | | | 0 |
| HOOCCH2CH(OH)COOH | − | − | 0 | 0 | 0 | 0 | + | | ++ | 0 |
| HOOCCH(OH)CH(OH)COOH | − | 0 | 0 | 0 | 0 | 0 | + | + | ++ | 0 |
| C6H10O8 | | | | | | | | | +++ | |
| HOOCCH2CH(SH)COOH | | | | | + | ++ | | | 0 | ++ |
| HOOCCH2—S—CH2COOH | | − | + | − | + | + | ++ | ++ | 0 | + |
| HOOCCH2CH(COOH)CH(OH)COOH | 0 | 0 | | 0 | | | | | | |
| HOOCCH2C(OH)(COOH)CH2COOH | 0 | 0 | 0 | 0 | + | + | ++ | | ++ | + |
| (C6H4)(OH)(COOH) | | | | ++ | ++ | ++ | ++ | | +++ | + |
| CH3(C═O)CH2(C═O)CH3 | + | | + | + | + | ++ | ++ | +++ | +++ | |
| C2H5O2N | 0 | − | + | | | ++ | ++ | | +++ | ++ |
| C3H7O2N | | | | | | ++ | ++ | | | ++ |
| C5H11O2N | | | | | | ++ | ++ | | | |
| C6H13O2N | | | | 0 | | | ++ | | | ++ |
| C9H11O2N | | | | | | | ++ | | | ++ |
| C3H7O2N | | | | | | + | | | | |
| C4H7O4N | | | | | | ++ | ++ | 0 | | ++ |
| C5H9O4N | | | | | | ++ | ++ | | | + |
| C9H11O3N | | | | ++ | ++ | ++ | ++ | | | + |
| C9H11O3N | | | | ++ | ++ | ++ | ++ | | | + |
| C9H11O3N | | | | ++ | ++ | ++ | ++ | | | + |
| C4H9O3N | | | | | + | | ++ | | | + |
| C5H10O3N2 | | | | | | ++ | ++ | | | ++ |
| C3H7O2NS | | | | | +++ | +++ | | | | |
| C6H12O4N2S2 | | | | | | ++ | +++ | | | ++ |
| C5H12O2N2 | | | | | | ++ | ++ | | | + |
| C6H14O2N2 | | | | | | ++ | ++ | | | + |
| C6H9O2N3 | | − | | + | ++ | ++ | ++ | + | | ++ |
| C11H12O2N2 | | | | | | | ++ | | | + |
| C3H8O5NP | + | + | ++ | ++ | ++ | ++ | ++ | ++ | +++ | ++ |
| C4H7O4N | 0 | + | | + | ++ | ++ | ++ | | ++ | ++ |
| C6H9O6N | + | + | | | | | ++ | ++ | | |
| C10H18O7N2 | | | | ++ | ++ | | ++ | | | ++ |
| C10H16O8N2 | + | ++ | ++ | ++ | ++ | ++ | ++ | | +++ | ++ |
| C3H6O3 | | | | | − | − | 0 | | | |
| H3O3B | − | − | | | | | | | ++ | |

Described below by way of example are the properties of inorganic, nitrogen- and sulphur-free organic acids, sugars, organic sulphur compounds, amino acids, chelate complexing agents and other compounds as complexing agents.

### Inorganic Complexing Agents:

The hydronium ion forms not-easily-dissolved complexes, especially with the rare earths. With all subgroup elements of the fourth period, also individual members of the boron group, both readily soluble and hard to dissolve compounds are formed. As an example, cobalt may be specified here.

Unprotected cobalt in water may carry out the following dissociation reactions:

Co²⁺+OH⁻ [symbol] CoOH⁺ pK=4.3

Co²⁺+2OH⁻ [symbol] Co(OH)₂ pK=8.4

Co²⁺+3OH⁻ [symbol] Co(OH)₃⁻ pK=9.7

Co²⁺+4OH⁻ [symbol] Co(OH)₄²⁻ pK=10.2

2Co²⁺+OH⁻ [symbol] (Co)₂OH³⁺ pK=2.7

4Co²⁺+4OH⁻ [symbol] ^{(Co)}₄(OH)₄ pK=25.6

Co²⁺+2OH⁻ [symbol] Co(OH)₂(s)↓ pK=14.9

If the solubility product of Co(OH)₂ is exceeded, the precipitation reaction predominates, since the activity of the solid is defined as 1 and is therefore no longer dependent on its concentration.

$$\begin{matrix}K = {10}^{14 \text{,} 9} \\ = \frac{a ( {Co ( OH )}_{2} )}{a ( {Co}^{2 +} ) \cdot {a ( {OH}^{-} )}^{2}} \\ = \frac{1}{a ( {Co}^{2 +} ) \cdot {a ( {OH}^{-} )}^{2}} a ( {Co}^{2 +} ) \cdot {a ( {OH}^{-} )}^{2} \\ = {10}^{- 14 \text{,} 9}\end{matrix}$$

However, before the solubility product of the cobalts is exceeded, the soluble cobalt hydroxide complexes reduce the concentration of the free Co²⁺ ions.

While the anion of hydrogen cyanide (CN⁻) and its complex compounds, which may also serve as ligands, do form very stable complexes with the subgroup elements of the fourth period, such complexes are however not anaerobically decomposable and therefore not suitable for the purposes of the invention involving anaerobic fermentation. The form thiocyanate (HCS⁻) however, which is closely related to hydrogen cyanide, may be used since the complexes it forms are not quite so stable.

The oxygen compounds of phosphorus complex bivalent cations to a high degree. Especially preferred here are polyphosphates, such as pyrophosphate and triphosphate. Pyrophosphate complexes magnesium and manganese very strongly, even in the presence of Zn²⁺, Fe²⁺, Ni²⁺ and Co²⁺, which are preferably bonded by the majority of complexing agents.

Thanks to its property as a Lewis acid, boric acid is a very good complexing agent for Fe³⁺. Bivalent ions such as Ca²⁺ and Mg²⁺ are complexed only with difficulty.

### Nitrogen- and Sulphur-Free Organic Acids:

The free volatile fatty acids (volatile fatty acids, VFA: formic acid, acetic acid, propionic acid, i-, n-butyric acid, i-, n-valerian acid, n-caproic acid) show only weak complexing properties. Cu²⁺ and Fe³⁺ are moderately complexed by VFA. Cu²⁺ is moderately complexed; the extent of complexing of VFA-Fe³⁺ complexes falls as chain length increases.

Modified short-chain hydroxy or ketofatty acids likewise show only weak tendencies to the formation of complexes. Hydroxyacetic acid (glycolic acid), 2-hydroxypropionic acid (lactic acid), oxoethanoic acid, oxopropionic acid (pyruvic acid, pyruvate) are partly formed in considerable amounts in the cell. They form complexes in small amounts with Cu²⁺, and also somewhat more poorly with Fe²⁺, Ni²⁺ and Co²⁺.

Oxalic acid is a moderate complexing agent with Fe²⁺, Ni²⁺, Co²⁺, Cu²⁺ and Zn²⁺ and a good complexing agent for Fe³⁺, however Ca²⁺ precipitates from the solution. Tartaric acid, malic acid and meso-malic acid have poor complexing properties for bivalent ions (except for Cu²⁺), but good complexing properties for trivalent ions (Fe³⁺, Al³⁺). Citric acid and to a somewhat lesser extent also iso-citric acid show good complexing properties for Co²⁺, Ni²⁺, Cu²⁺ and Fe³⁺. Salicylic acid is a good complexing agent for Zn²⁺, a very good complexing agent for Mn²⁺, Co²⁺, Ni²⁺, Cu²⁺ and an excellent complexing agent for Fe³⁺.

Gluconic acid is moderate complexing agent for Ni²⁺ complexes.

### Sugars:

Of the sugars, galacturonic acid, the monomer of polygalacturonic acid, a basic building block of pectin, may be cited as a noteworthy complexing agent. It is able, selectively, to complex Fe²⁺ very well. Other hexoses and pentoses such as e.g. glucose, galactose or arabinose have no great tendencies to form complexes.

### Organic Sulphur Compounds:

Nitrogen- and sulphur free organic acids—such as described above, in which an oxygen atom was replaced by a sulphur atom, have much better complexing properties. Thus e.g. mercaptoacetic acid (thio-glycol acid) and mercaptopropionic acid (thio-lactic acid) are good complexing agents for Mn²⁺, very good for Fe²⁺, Co²⁺ and excellent complexing agents for Fe³⁺ and Zn²⁺. Mercaptomalic acid differs from malic acid in its complexing spectrum, in that it complexes Ni²⁺, Zn²⁺ well, and to a lesser extent also Co²⁺. In contrast to the organic sulphur compounds referred to earlier, thio-diacetic acid contains no —SH group, but instead an —S ether group. It complexes Fe²⁺, Co²⁺, Ni²⁺, Zn²⁺ well, Cu²⁺ and Al³⁺ very well, but not Fe³⁺.

### Amino Acids:

Amino acids are to some extent excellent complexing agents. They are by nature biologically decomposable or may at least be taken up by the cell and utilised. The amino acid glycine shows for Ca²⁺ poor and for Mg²⁺ moderate complexing properties. Co²⁺, Ni²⁺, Cu²⁺ and Zn²⁺ are complexed very well, and Fe³⁺ is complexed extremely well. Alanine and valine show similar complexing properties. They complex Ni²⁺, Cu²⁺ and Zn²⁺ very well. Leucine complexes Mn²⁺ only moderately, but Cu²⁺ and Zn²⁺ very well. For phenylalanine, very good complexing properties are known for Cu²⁺ and Zn²⁺. In the case of beta-alanine, good complexing properties are known only for Ni²⁺. Aspartic acid complexes Ni²⁺, Cu²⁺ and Zn²⁺ very well, but Al³⁺ only moderately. Glutamic acid, the salt of which is also known as a flavour enhancer, complexes Ni²⁺, Cu²⁺ very well, but Zn²⁺ not so well. Die ortho-, meta- and para-isomers of tyrosine show very similar properties with regard to complexing. They complex Zn²⁺ well, Mn²⁺, Ni²⁺, Co²⁺ and Cu²⁺ very well. Threonine exhibits good complexing properties for Co²⁺ and Zn²⁺, while Cu²⁺ is complexed very well. Glutamine shows very good complexing properties for Ni²⁺, Cu²⁺ and Zn²⁺. Cysteine shows the best complexing properties of all amino acids. Especially Co²⁺ and Ni²⁺ are complexed extremely well by cysteine. Also in its oxidised form, the disulphide cystine is excellent at holding Cu²⁺ in solution. Ni²⁺ and Zn²⁺ are also always very well complexed. The amino acid ornithine, which does not occur in proteins, and lysine exhibit similar complexing properties. They are very good at forming complexes with Ni²⁺ and Cu²⁺ complexes, while Zn²⁺ is complexed well. Histidine shows poor complexing properties for Ca²⁺, good for Mn²⁺ and Al³⁺ and very good for Co²⁺, Ni²⁺, Cu²⁺ and Zn²⁺. Tryptophan shows very good complexing properties for Cu²⁺ and good for Zn²⁺. The amino acids arginine, asparagine, isoleucine, methionine and serine, also the non-proteinogenic amino acids homo-cysteine and homo-serine are also able to complex metals.

Dipeptide and tripeptide also have very good complexing properties (e.g. L-valyl-L-valine for Ni²⁺), but these compounds are more expensive than simple amino acids.

### Chelate Complexing Agents:

Chelate complexing agents are generally tertiary amines. Their most prominent representatives are EDTA (ethylenediaminetetraacetic acid), which complexes Mg²⁺ well, Ca²⁺, Fe²⁺, Mn²⁺, Co²⁺, Ni²⁺, Cu²⁺, Zn²⁺ very well and Fe³⁺ extremely well, and NTA (nitrilotriacetic acid), which has a similar complexing spectrum and identical priorities. EDTA is not anaerobically decomposable and NTA is carcinogenic. But in addition there is a whole range of further chelate complexing agents which do not have these drawbacks. Ethylenediamine dibernstein acid (EDDS) has isomers, which are biologically decomposable. Ethylendiimine diacetic acid (EDDA) complexes Co²⁺ and Zn²⁺ very well, and Mn²⁺ well. Ethyleneglycol tetraacetic acid (EGTA) shows good complexing behaviour similar to EDTA, but has greater affinities to Ca²⁺ and Mg²⁺.

### Other Compounds:

Other compounds, such as the compound acetylacetone, complex—through the keto group—Mg²⁺, Mn²⁺, Fe²⁺, Co²⁺ moderately well, Ni²⁺, Cu²⁺ well and Fe³⁺ and Al³⁺ extremely well. Orotic acid, a heterocyclic non-aromatic with two nitrogen atoms is also able to complex Co²⁺, Ni²⁺ and Cu²⁺. While n-phosphomethylglicine is certainly a complexing agent with a very broad spectrum, it inhibits the aromatic amino acid synthesis and is not suitable as complexing agent for addition to a bioreactor. There are also known substitute materials such as zeolites, which act as molecular sieves and may also be used to improve the bio-availability of trace elements.

According to the invention complexing agents are used which are resorbed by microorganisms, preferably anaerobic bacteria, wherein (1) the trace elements are transferred in complexed form across the cell membrane and then (2) the trace elements are released in the cell. The latter may be effected, for example, by a consecutive reaction of the complexing agent, by oxidation or reduction of the trace elements, by the pH-shift on crossing the cell wall or through the biological decomposition of the complexing agent. In a bacterial process such as the biogas process the transfer of the trace elements takes place in complexed form across the bacterial cell wall and the cell membrane into the cytosol of the cell, where the trace element is released.

In one embodiment of the solution according to the invention, at least one of the complexing agents is biologically decomposable; if necessary all complexing agents are anaerobically decomposable.

Suitable complexing agents which meet the specified criteria according to the invention are known and to some extent are available commercially. Examples of preferred complexing agents according to the invention are: oxocarboxylic acids, for example β-oxocarboxylic acids such as acetoacetate or α-oxocarboxylic acids such as pyruvic acid and its respective salts; acetylacetone; orotic acid; simple amino acids, for example alanine, valine, cystine, phenylalanine, aspartic acid, glutamic acid, leucine, threonine, tryptophan or glycine, also ortho-, meta- and para-isomers of tyrosine; dipeptide, tripeptide; polymethine dyes such as for example catechol (also known as catechin); citric acid and its salts, iso-citric acid and its salts; salicylic acid; chelate complexing agents such as tertiary amines, for example diethylenetriaminepentaacetic acid (DTPA), hydroxyethylenediaminetriacetic acid (HEDTA), ethylenediamine dibernstein acid (EDDS), ethylendiiminodiacetic acid (EDDA); dicarboxylic acids, such as for example malonic acid, tartaric acid, malic acid, meso-malic acid or oxalic acid and their salts; hydroxycarboxylic acids, such as for example lactic acids and their salts; modified cyclodextrane; galacturonic acid; mercaptoacetic acid (thioglycolic acid), mercaptoproprionic acid (thiolactic acid), mercaptomalic acid, thiodiacetic acid; boric acid, phosphorus acid, salts of phosphorus acid such as (hydroxy-) phosphonate, phosphoric acid, salts of phosphoric acid such as (hydroxy-) phosphate, polyphosphate, for example di- and triphosphate; oligopeptides such as the iron-binding siderophores such as enterochelin; and zeolites.

The combination according to the invention of two or more complexing agents in the trace element solution may for example be comprised of these complexing agents.

Advantageously the complexing agents are selected from: acetoacetate, simple amino acids, pyruvic acid, catechole, citric acid, salts of citric acid, tertiary amine, malonic acid, lactic acid, modified cyclodextrane, oxalic acid, phosphorous acid, salts of phosphorous acid, phosphoric acid, salts of phosphoric acid, polyphosphate, siderophores, tartaric acid and zeolites.

In a preferred embodiment of the invention the trace element solution contains as complexing agent at least one tertiary amine, for example EDTA, NTA, EDDS, EDDA; and at least one complexing agent chosen from at least one inorganic complexing agent, at least one nitrogen- and sulphur-free organic acid, at least one amino acid and mixtures thereof.

The inorganic complexing agent is preferably an oxygen compound of phosphorus. The nitrogen- and sulphur-free organic acid may be selected from, for example, citric acid, iso-citric acid, salicylic acid, gluconic acid and mixtures thereof.

In an especially preferred embodiment of the invention the trace element solution includes as complexing agent EDTA and an oxygen compound of phosphorus, in particular at least a phosphoric acid, phosphorus acid or its salts, for example polyphosphates such as pyrophosphate.

In another preferred embodiment of the invention the trace element solution includes as complexing agent EDTA and citric acid or a salt of citric acid.

In a further embodiment of the invention the trace element solution includes as complexing agent at least one oxygen compound of phosphorus and at least one complexing agent selected from tertiary amines, amino acids, citric acid, salts of citric acid and mixtures thereof.

Especially preferred is a trace element solution, which contains as complexing agent at least one oxygen compound of phosphorus and at least one amino acid. Highly suitable according to the invention is, for example, also a trace element solution which includes at least one oxygen compound of phosphorus and a citric acid or its salt. Tertiary amines are not included in these solutions, but may be added if required.

If amino acids are used as complexing agents according to the invention, then at least one simple amino acid may be selected which in particular complexes cobalt, nickel and/or zinc well; for example, glycine, alanine, valine, ortho-, metha- and para-isomers of tyrosine, threonine, cysteine or histidine.

If at least one oxygen compound of phosphorus is used as complexing agent according to the invention, then for example a phosphoric acid, phosphorus acid and salts thereof, in particular polyphosphates such as, for example pyrophosphate or triphosphate may be used. Mixtures of different phosphates, with polyphosphates being especially preferred, may also be used advantageously.

The use of phosphoric acid, polyphosphates and phosphates as complexing agents is advantageous, since in this case the micronutrient phosphorus is given as an additive at the same time. Therefore, in using phosphoric acid or phosphates, depending on the phosphorus requirement of the process concerned, they may be added in suitable excess amounts to the trace element solution or the fermenter.

To ensure additional stability, for example against impact loads during fermentation, it is possible to provide in the trace element solution, alongside the aforementioned two or several complexing agents, an additional strong complexing agent, for example from the group of the tertiary amines, such as diethylenetriaminepentaacetic acid (DTPA), ethylenediaminetetraacetic acid (EDTA), hydroxyethylenediaminetriacetic acid (HEDTA) and/or, if necessary, nitrilotriacetic acid (NTA) on top of the two or more different complexing agents. However, trace element solutions according to the invention may also be produced without tertiary amines.

A further exemplary combination of complexing agents for trace element solution according to the invention is ethylenediaminetetraacetic acid (EDTA), citric acid and catechol. If necessary this trace element solution may also include further complexing agents. Where applicable EDTA may be replaced by an anaerobically decomposable, strong complexing agent. In a preferred embodiment of this kind, the trace element solution comprises the combination of at least one phosphoric acid or phosphorous acid or its salts, e.g. a phosphate, in particular polyphosphate, and complexing agent from the group comprised of galacturonic acid, acetylacetonate and amino acids.

For certain uses of the invention it may be advantageous to add to the trace element solution neither EDTA, nor NTA or n-phosphomethylglicine, since EDTA is not anaerobically decomposable, NTA is carcinogenic and n-phosphomethylglicine inhibits the aromatic amino acid synthesis.

The trace elements, also described as trace metals or micronutrients, include iron (Fe), nickel (Ni), cobalt (Co), selenium (Se), tungsten (W), lead (Pb), copper (Cu), cadmium (Cd), molybdenum (Mo), tungsten (W), vanadium (V), manganese (Mn), boron (B) and zinc (Zn). The trace element solution of the invention includes at least one of these elements. The composition of the trace element solution and the amount of the element concerned will depend on the substrate used and the microorganisms of the particular fermentation. For biogas processes the trace element solution preferably includes at least molybdenum, cobalt, boron and where applicable nickel. The latter trace element solution is advantageous especially for maize substrates. In biogas processes molybdenum, nickel and cobalt may be added to the fermenter in relatively high concentrations, which enhances significantly the performance and efficiency of fermentation. Cobalt, nickel and manganese are only very weakly soluble metals (in particular in comparison with magnesium and calcium), and for this reason the increase in bio-availability of these metals due to the trace element solution according to the invention is especially advantageous, since in particular cobalt and nickel, but also manganese, are essential for methanoganesis.

In addition to trace elements and the complexing agent the solution according to the invention may also include other alkaline, alkalkine-earth and heavy metals; enzymes, vitamins, amino acids, fatty acids, carbon sources, nitrogen compounds and other nutrients, which are advantageous for metabolism of the microorganisms in the bioreactor.

The invention also relates to the use of the trace element solution according to the invention for a biogas process.

A trace element solution comprising at least one, preferably two or more, of the complexing agents described above, is also useful for other kinds of anaerobic fermentation besides biogas processes, with neutral or weak acid pH value, in which trace elements may precipitate or form difficult-to-dissolve complexes in the presence of sulphide ions.

Suitable as starting substrate for biogas processes according to the invention are for example: fermentable residues such as sewage sludge, bio-waste or leftover food; fertilisers such as liquid or solid manure; also regrowing energy plants such as maize, cereals or grass.

Use of the trace element solution according to the invention is advantageous in biogas processes with monosubstrates such as industrial effluent or plant raw materials.

If necessary it is also possible to add to the biogas process, in addition to the trace element solution according to the invention, at least one complexing agent or a mixture of complexing agents according to the invention. In biogas processes, which convert iron-, magnesium- or calcium-rich substrates, for example effluent from papermills, it is advantageous to add a surplus of the complexing agent according to the invention, in order to prevent the phenomenon described in connection with Table 2 of the displacement of cobalt, nickel and zinc by magnesium and/or calcium. Preferably for this purpose a mixture of complexing agents according to the invention is added to the biogas process, on top of the trace element solution according to the invention.

Thus the trace element solution may be used for biogas processes which operate solely with monosubstrates based on vegetable biomass, for example from agricultural production. Such a process requires no co-substrates in the form of animal excrement, for example liquid manure, stable manure or dried excrement. The monosubstrate for fermentation may also be a mixture of different types of preparations of the same substrates, e.g. a mixture of maize silage, maize grains and fresh maize. As an alternative to this it is also possible of course for mixtures of different vegetable substrates, e.g. of maize and grass, to be fermented.

Suitable as monosubstrates are vegetable products and/or waste. These include cut grass, silage, energy crops, als “continuously growing raw materials” (NAWRO) designated plants, storage residues, harvest residues or vegetable waste. Examples of plants suitable as substrates: maize, rye, grass, turnips, sunflowers and rapeseed. Industrial effluents, as for example from papermills, also represent monosubstrates.

In tests with maize silage it was found, surprisingly, that the fermentability of the substrate was improved by the addition of a trace element solution according to the invention. Moreover, through the further addition of phosphate to the substrate of maize silage, a marked increase in gas production was obtained, while the hydraulic retention time of the substrates was reduced. By this means it was possible to increase the volumetric loading of the fermenters by around tenfold, from roughly 1.5 kg to around 10 kg_{oTM}/(m³ d). In the vegetable material, organically bound phosphorus and trace elements are available for the methane fermentation to only a limited extent. Consequently the conversion rate of the bacteria involved in the fermentation may be increased significantly through addition of the trace element solution, thereby improving utilisation of the vegetable substrates used and by this means reducing the fermentation residue in the bioreactor.

The trace element solution according to the invention is especially advantageous for Mg²⁺- and/or Ca²⁺-rich fermentation substrates since, due to the at least two complexing agents of varying strength, adequate solubility and/or bio-availability of the weakly soluble micronutrients such as cobalt, nickel and manganese is provided, despite the increased solubility of magnesium and, where applicable calcium, under the conditions of the biogas fermentation.

The invention also includes a process for the production of biogas in a biogas plant, in which during fermentation a trace element solution is fed into the fermenter for biogas production and this trace element solution comprises at least one trace element and at least one of the complexing agents described above. The trace element solutions described above with two or several complexing agents are preferred.

Where applicable, the trace elements and the complexing agents may also be provided in dry, e.g. lyophilised or powder form, and only brought into solution immediately before being fed into the fermenter. The dosing of the trace element solution into the fermenter may be batchwise, discontinuous or continuous.

The invention is illustrated below by Figures and examples which do not restrict the invention, and showing in:

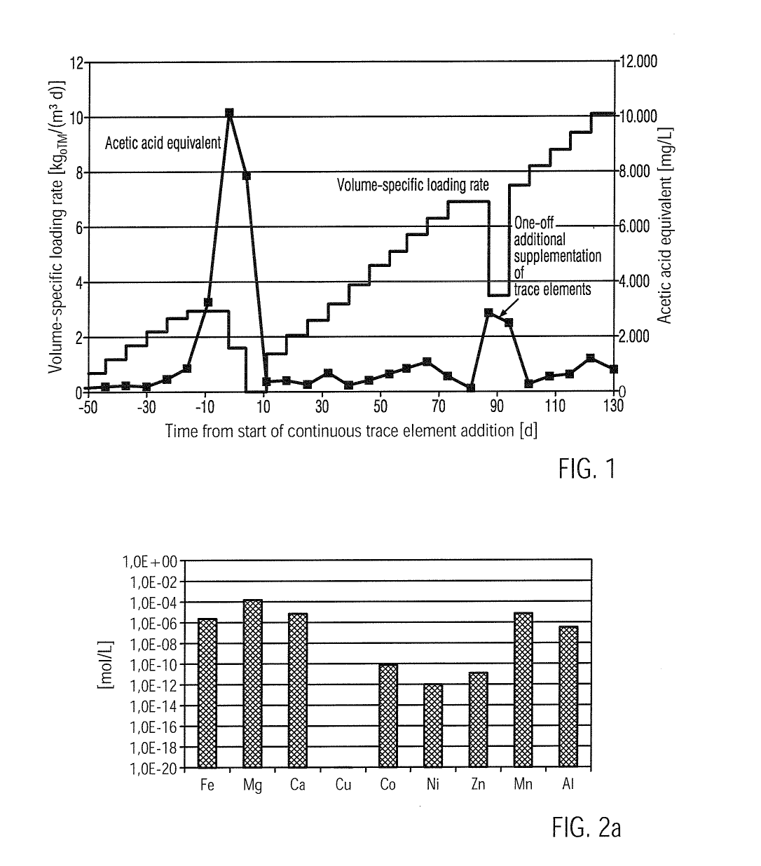

FIG. 1 Addition of a complexed trace element solution to a 500 m³ biogas reactor with maize silage according to Example 3. The addition starts with the beginning of acidification of the reactor and a volumetric loading of 3 kg_{oTM}/(m³ d). Through the addition of bio-available trace elements, the volumetric loading may be increased to 10 kg_{oTM}/(m³ d), without volatile fatty acids accumulating in the reactor,

FIG. 2 Table 2 data:
(a) Concentration of trace elements in the fermentation residue complexed with 50% NTA and a trace element composition according to Table 5,
(b) Concentration of trace elements in the fermentation residue complexed with 50% NTA and one hundred times the trace element feed amount of Table 5.
(c) Concentration of trace elements in fermentation residue complexed with 50% NTA and a normal amount of Fe of Table 5 and one hundred times the amount of the other trace elements of Table 5.

FIG. 3 Table 3 data:
(a) Concentration of the trace elements in the fermentation residue complexed with 50% NTA after addition of interfering agents (500% Fe(III)),
(b) Concentration of trace elements in the fermentation residue complexed according to the invention (100% EDTA, 1000% phosphoric acid mixture) after addition of interfering agents (500% Fe(III)

### EXAMPLE 1

### Complexing of the Trace Element Solution of DSMZ Medium 141

The composition of the trace element solution is set out in Table 5. Also of note here is the fact that according to references the concentration of ions which may be precipitated by sulphide is distinctly higher than the concentration of the complexing agent NTA. In the use of this trace element solution, also as expected, a fine sediments forms, as soon as a sulphur-based (Na₂S; Na₂S₂O₃) reduction agent is added. This may be prevented by a suitable addition according to the invention of complexing agents e.g. 15 mmol/L pyrophosphate, 0.2 mmol/L galacturonic acid, 0.4 mmol/L cysteine, 0.05 mmol/L acetylacetonate and 0.3 mmol/L leucine.

TABLE 5
Table 5: Composition of the trace element solution
of DSMZ medium 141 for a methaneogenic archaeon

| | m [g/L] | c [mmol/L] |
|---|---|---|
| NTA | 1.5000 | 7.853 |
| MgSO₄ × 7 H₂O | 3.0000 | 13.717 |
| MnSO₄ × 2 H₂O | 0.5000 | 2.277 |
| NaCl | 1.0000 | 21.739 |
| FeSO₄ × 7 H₂O | 0.1000 | 0.199 |
| CoSO₄ × 7 H₂O | 0.1800 | 0.339 |
| CaCl₂ × 2 H₂O | 0.1000 | 0.500 |
| ZnSO₄ × 7 H₂O | 0.1800 | 0.306 |
| CuSO₄ × 5 H₂O | 0.0100 | 0.022 |
| KAl(SO₄)₂ × 12 H₂O | 0.0200 | 0.032 |
| H₃BO₃ | 0.0100 | 1.429 |
| Na₂MoO₄ × 2 H₂O | 0.0100 | 0.087 |
| NiCl₂ × 6 H₂O | 0.0250 | 0.047 |
| Na₂SeO₃ × 5 H₂O | 0.0003 | 0.002 |

### Example 2

Shown in Table 6 is an exemplary composition of a trace element solution according to the invention. Used as first strong complexing agent is EDTA and as second complexing agent a mixture of phosphorous acids. If the substrate of the biogas fermentation is an effluent, e.g. of a papermill, then the solution may be added, for example, at a ratio of 1:1000 to the substrate. If the substrate is a waste or vegetable raw material, the solution may be added to the substrate at a ratio of, for example, 1:100.

TABLE 6

| Element | mmol/L |
|---|---|
| Mo | 0.42 |
| Ni | 1.12 |
| Se | 0.08 |
| W | 0.90 |
| Mn | 0.80 |
| Co | 1.00 |
| Zn | 0.74 |
| Cu | 0.59 |
| B | 1.64 |
| Fe | 4.60 |

| Complexing | | |
|---|---|---|
| agent | mmol/L | mg/L |
| EDTA | 7.2 | 2102 |
| H4P2O7 | 7.2 | 1282 |
| H6P4O13 | 7.2 | 2434 |
| H4P4O12 | 7.2 | 2304 |
| H3PO2 | 7.2 | 475 |
| H3PO3 | 7.2 | 590 |

### Example 3

### Dry Fermentation of Maize Silage in a 500 kW Plant

In a plant designed in accordance with DE102005041798, maize silage is feremented and converted into biogas. At the start of feeding, a volume-specific loading rate of 0.75 kg_{oTM}/(m³ d) is set and the feed rate per week is increased by 0.5 kg_{oTM}/(m³ d). On reaching a volume-specific loading rate of 3 kg_{oTM}/(m³ d), the acids in the reactor begin to increase—a sign that the anaerobic biomass in the reactor is overloaded. The increase in feeding is suspended for time being, but the rise in acids continues. A commercially available trace element solution, complexed with two complexing agents of different strength according to the method described in the invention, is now added to the reactor. The acids thereupon decline within 10 days and feeding is continued. Just 90 days from the start of continuous addition of trace elements, the acids increase again. The volume-specific loading rate is meanwhile 7 kg_{oTM}/(m³ d). The feed rate is thereupon halved for one week and ten times the daily dose of trace elements is added. After a week, feeding is again reset to the old value and further increased. The reactor reaches its design specification at 10 kg_{oTM}/(m³ d). At 1000 mg/L the acid concentration lies below the upper limit of 2000 mg/L for the EEC technology bonus. Only the addition of the complexed trace element solution allows the increase in the volume-specific loading rate of 5 (prior art) to 10 kg_{oTM}/(m³ d). The dry fermentation was carried out by a known process (Conclusions of the Biogas-measuring Programme, 2005, Special Agency for Regrowing Raw Materials, Section 7.3).

The addition of the complexed trace element solution according to the invention to a 800 m³ biogas reactor with maize silage is shown in FIG. 1. The addition commences with the start of acidification of the reactor at a volumetric loading rate of 3 kg_{oTM}/(m³ d). Through the addition of bio-available trace elements, it is possible to increase the volumetric loading rate to 10 kg_{oTM}/(m³ d), without volatile fatty acids accumulating in the reactor.

The fermentation residue was suitable as fertiliser (As<0.1 mg/kg TM, Pb 2.42 mg/kg TM, Ca 0.28 mg/kg TM, Cr 8.96 mg/kg TM, N±6.05 mg/kg TM, Hg 0.08 mg/kg TM, TI <0.2 mg/kg TM, Se<0.5 mg/kg TM, Cu 3 mg/kg FM, Zn 10 mg/kg FM, B 0.8 mg/kg FM, Co 0.072 mg/kg FM; TM=dry matter, FM=fresh matter).

1-21. (canceled)

22. A method for the production of biogas in a biogas fermenter, comprising the steps of:
providing a vegetable biomass as a substrate for the production of biogas; and
adding a trace element solution to the biomass, wherein the trace element solution comprises at least one trace element,
a first complexing agent which complexing constant (pK value) for Fe³⁺ is greater than the complexing constant (pK value) of said complexing agent for Co²⁺ or Ni²⁺; and
a second complexing agent different from said first complexing agent which complexing constant (pK value) for Co²⁺ and Ni²⁺ are at least 5,
wherein the pK value of the complexing constant: pK=−IgK=−Ig([MLₙ]/[M][L]ⁿ), wherein K is the complexing constant, [MLₙ] is the molar equilibrium concentration of the trace element complex, [M] is the molar concentration of the free trace element, [L] is the molar equilibrium concentration of the complexing ligand and n is the number of ligands bound in the complex.

23. The method according to claim 22, wherein the complexing constants (pK value) for Fe³⁺ of the first complexing agent is at least 10.

24. The method according to claim 22, wherein the complexing constants (pK values) for Co²⁺ and Ni²⁺ of the second complexing agent are greater than 10.

25. The method according to claim 22, wherein the complexing constant (pK value) of the second complexing agent for Fe³⁺ is smaller than the complexing constant (pK value) of the first complexing agent for Fe³⁺.

26. The method according to claim 22, wherein the first and the second complexing agents are present in at least equimolar amounts to the at least one trace element.

27. The method according to claim 22, wherein the trace element solution comprises at least Co²⁺, Ni²⁺ and Mn²⁺.

28. The method according to claim 22, wherein the trace element solution comprises at least Mo²⁺, Co²⁺ and B.

29. The method according to claim 22, wherein the vegetable biomass is selected from the group consisting of cut grass, silage, energy crops, storage residues, harvest residues and vegetable waste.

30. The method according to claim 22, wherein the vegetable biomass is selected from the group consisting of maize, rye, grass, turnips, sunflowers and rapeseed.

31. The method according to claim 22, wherein the trace element solution comprises more than two different complexing agents.

32. The method according to claim 22, wherein
(a) at least one complexing agent is a tertiary amine; and
(b) at least one complexing agent is selected from the group consisting of inorganic complexing agents, nitrogen- and sulphur-free organic acids, and mixtures thereof.

33. The method according to claim 30, wherein the inorganic complexing agent is an oxygen compound of phosphorus.

34. The method according to claim 31, wherein the trace element solution comprises two or more different oxygen compounds of phosphorus.

35. The method according to claim 22, wherein
(a) at least one complexing agent is an oxygen compound of phosphorus; and
(b) at least one complexing agent is selected from the group consisting of tertiary amines, citric acid and mixtures thereof.

36. The method according to claim 35, wherein the trace element solution comprises two or more different oxygen compounds of phosphorus.

37. A method for the production of biogas in a biogas fermenter, comprising the steps of:
providing a substrate for the production of biogas; and
adding a trace element solution to the substrate, wherein the trace element solution comprises at least the trace elements Mo²⁺, Co²⁺ and B,
a first complexing agent, and
a second complexing agent different from said first complexing agent.

38. The method according to claim 37, wherein the first and the second complexing agents are present in at least equimolar amounts to the at least one trace element.

39. The method according to claim 37, wherein the substrate is a maize preparation.

40. The method according to claim 37, wherein the complexing constants (pK values) of the complexing agents for the trace elements are at least 5.

41. The method according to claim 37, wherein not more than 30 mL trace element solution per ton of dry substance of the substrate are added.

42. The method according to claim 37, wherein
(a) at least one complexing agent is a tertiary amine; and
(b) at least one complexing agent is selected from the group consisting of inorganic complexing agents, nitrogen- and sulphur-free organic acids, and mixtures thereof.

43. The method according to claim 42, wherein the inorganic complexing agent is an oxygen compound of phosphorus.

44. The method according to claim 43, wherein the trace element solution comprises two or more different oxygen compounds of phosphorus.

45. The method according to claim 37, wherein
(a) at least one complexing agent is an oxygen compound of phosphorus; and
(b) at least one complexing agent is selected from the group consisting of tertiary amines, citric acid and mixtures thereof.

46. The method according to claim 45, wherein the trace element solution comprises two or more different oxygen compounds of phosphorus.

47. The method according to claim 37, wherein the trace element solution further comprises Ni²⁺.

48. A method for the production of biogas in a biogas fermenter comprising the steps of:
providing a substrate for the production of biogas; and
adding a trace element solution to the substrate, wherein the trace element solution comprises:
at least one trace element;
a first complexing agent which complexing constant (pK value) for Fe³⁺ is at least 10; and
a second complexing agent different from said first complexing agent which complexing constant (pK value) for Co²⁺ and Ni²⁺ are at least 5,
wherein the first and the second complexing agents are present in at least equimolar amounts to the at least one trace element and the pK value of the complexing constant: pK=−IgK=−Ig([MLₙ]/[M][L]ⁿ), wherein K is the complexing constant, [MLₙ] is the molar equilibrium concentration of the trace element complex, [M] is the molar concentration of the free trace element, [L] is the molar equilibrium concentration of the complexing ligand and n is the number of ligands bound in the complex.

49. The method according to claim 48, wherein the trace element solution is fed into a fermenter for biogas production during fermentation.

50. The method according to claim 48, wherein
(a) at least one complexing agent is a tertiary amine; and
(b) at least one complexing agent is selected from the group consisting of inorganic complexing agents, nitrogen- and sulphur-free organic acids, and mixtures thereof.

51. The method according to claim 50, wherein the inorganic complexing agent is an oxygen compound of phosphorus.

52. The method according to claim 50, wherein the nitrogen- and sulphur-free organic acid is selected from the group consisting of citric acid, iso-citric acid, salicylic acid, gluconic acid and mixtures thereof.

53. The method according to claim 48, wherein
(a) at least one complexing agent is an oxygen compound of phosphorus; and
(b) at least one complexing agent is selected from the group consisting of tertiary amines, citric acid and mixtures thereof.

54. The method according to claim 53, wherein the trace element solution comprises two or more different oxygen compounds of phosphorus.

55. The method according to claim 48, wherein the substrate is a monosubstrate.

56. The method according to claim 48, wherein the substrate is a vegetable product.

57. The method according to claim 48, wherein the trace element solution comprises at least Co²⁺, Ni²⁺ and Mn²⁺ and/or B.

58. The method according to claim 48, wherein the first and second complexing agents are neither aminoacids nor a peptide.
